(19) United States
(12) Patent Application Publication
(10) Pub. No.: US 2021/0154215 A1
(43) Pub. Date: May 27, 2021

(54) Antifungal Cuticle Oil Composition

(71) Applicant: Zenvision Pharma LLP, Mumbai (IN)

(72) Inventors: Sivakumar Venkata Bobba, Navi Mumbai (IN); Bhimrao Jadhav, Navi Mumbai (IN); Dhananjay Shinde, Navi Mumbai (IN); Sunil Pophale, Navi Mumbai (IN)

(21) Appl. No.: 15/734,611

(22) PCT Filed: May 24, 2019
(86) PCT No.: PCT/IB2019/054329

§ 371 Date: Dec. 3, 2020

(30) Foreign Application Priority Data
Jun. 4, 2018 (IN) 201821020765

(51) Int. Cl. A61K 31/69; A61K 31/454; A61K 31/137; A61K 9/00; A61K 47/44; A61K 47/14

(52) U.S. Cl. CPC A61K 31/69; A61K 31/454; A61K 47/14; A61K 9/0014; A61K 47/44; A61K 31/137

(57) ABSTRACT

The present invention relates to antifungal cuticle oil composition comprising Efinaconazole or Tavaborole or Terbinafine or salt thereof in the treatment of onychomycosis. The topical antifungal cuticle oil according to present invention provides better penetration and application at affected site results in better efficacy and patient compliance in the treatment of onychomycosis. The said composition also combat fungus of nail, cuticle, nail bed area and skin surrounding nail, protect and nourish the nails which got brittle, inflamed, painful and white to yellow in onychomycosis.

### FIELD OF THE INVENTION

The present invention relates to antifungal cuticle oil composition comprising Efinaconazole or Tavaborole or Terbinafine or salt thereof in the treatment of onychomycosis.

### BACKGROUND OF THE INVENTION

Onychomycosis, also known as Tinea unguium, is a disease of the nail caused by yeast, dermatophytes, or other molds and represents approximately 50% of all nail disorders. Toenail infection accounts for approximately 80% of onychomycosis incidence, while fingernails are affected in about 20% of the cases. Dermatophytes are the most frequent cause of nail plate invasion, particularly in toenail onychomycosis.

The medications available for the treatment of fungal infections including the oral and topical use of antibiotics (e.g. Nystatin and Amphotericin B), imidazole anti-fungal agents such as miconazole, bifonazole, clotrimazole, fluconazole, econazole, ketoconazole, tioconazole and sulconazole, non-imidazole antifungal agents such as the allylamine derivatives terbinafine, naftifine and the benzylamine butenafine, triazole antifungal agents such as efinaconazole, fluconazole, fosfluconazole, terconazole and itraconazole, others include tavaborole, ciclopirox, griseofulvin, amorolfine or tolnaftate.

However, onychomycosis has proven to be resistant to most treatments. Nail fungal infections reside in an area difficult to access by conventional topical treatment and anti-fungal drugs cannot readily penetrate the nail plate to reach the infection sites under the nail. Therefore, onychomycosis has traditionally been treated by oral administration of anti-fungal drugs; however, this is undesirable due to the potential for side effects of such drugs, in particular those caused by the more potent anti-fungal drugs such as itraconazole and ketoconazole.

Efinaconazole is an azole antifungal with a chemical name of ((2R,3R)-2-(2,4-difluorophenyl)-3-(4-methylenepiperidin-1yl)-1-(1H-1,2,4-triazol-1-yl)butan-2ol) and its molecular weight is 348.39 g/mol. Its empirical formula is C₁₈H₂₂F₂N₄O. Efinaconazole is represented by compound of structural formula I.

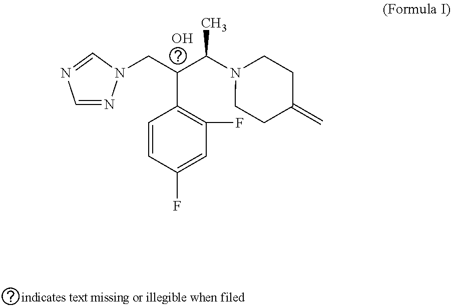

Efinaconazole is white to pale yellow crystals or crystalline powder. It is practically insoluble in water.

Efinaconazole topical solution of Dow Pharmaceutical Sciences has been approved in USA on Jun. 6, 2014 under the trade name JUBLIA® and is available in the strength of 10%. The product is indicated for the topical treatment of onychomycosis of the toenail(s) due to Trichophyton rubrum and Trichophyton mentagrophytes.

Tavaborole, is an oxaborole antifungal with the chemical name of 5-fluoro-1,3-dihydro-1-hydroxy-2,1-benzoxaborole and its molecular weight is 151.93 g/mol. Its empirical formula is C₇H₆BFO₂. Tavaborole is represented by compound of structural formula II.

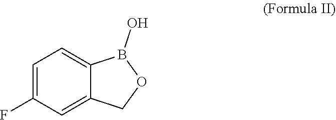 [SMILES: OB1OCc2cc(F)ccc21]

Tavaborole is a white to off-white powder. It is slightly soluble in water and freely soluble in ethanol and propylene glycol.

Tavaborole topical solution of Anacor Pharmaceuticals Inc has been approved in USA on Jul. 7, 2014 under the trade name KERYDIN® and is available in the strength of 5%. The product is indicated for the treatment of onychomycosis of the toenails due to Trichophyton rubrum or Trichophyton mentagrophytes.

Terbinafine hydrochloride is an allylamine antifungal with a chemical name of (E)-N-(6,6-dimethyl-2-hepten-4-ynyl)-N-methyl-1-naphthalenemethanamine hydrochloride and its molecular weight is 327.90 g/mol. Its empirical formula is C₂₁H₂₆ClN. Terbinafine hydrochloride is represented by compound of structural formula III.

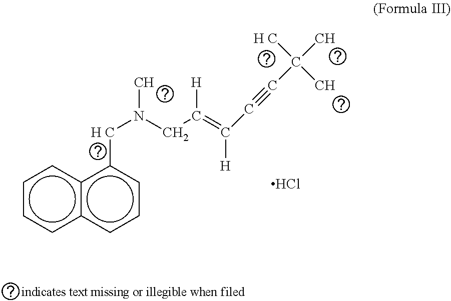

Terbinafine hydrochloride is a white to off-white fine crystalline powder. It is freely soluble in methanol and methylene chloride, soluble in ethanol, and slightly soluble in water.

Terbinafine hydrochloride tablet of Novartis Pharmaceuticals has been approved in USA on Mar. 10, 1996 under the trade name LAMISIL and is available in the strength of eq 250 mg base. The product is indicated for the treatment of onychomycosis of the toenail or fingernail due to dermatophytes (Tinea unguium).

U.S. Pat. No. 5,962,476 discloses compound Efinaconazole or salt thereof specifically.

U.S. Pat. No. 8,039,494 discloses alcohol based Efinaconazole topical composition for the treatment of onychomycosis. The said patent does not disclose or teach cuticle oil composition of Efinaconazole or Tavaborole or Terbinafine or salt thereof.

U.S. Pat. No. 5,880,188 discloses compound Tavaborole or salt thereof generically.

U.S. Pat. No. 9,566,289 discloses alcohol based Tavaborole topical composition for the treatment of onychomycosis. The said patent does not disclose or teach cuticle oil composition of Efinaconazole or Tavaborole or Terbinafine or salt thereof.

US2016175335 discloses pharmaceutical composition comprising Efinaconazole, a boron-containing antifungal agent (Tavaborole) or salts thereof and one or more pharmaceutically acceptable excipients for treating fungal infections such as onychomycosis. US2016175335 discloses topical solution, gel, cream, lotion, tincture or ointment composition of Efinaconazole and boron-containing antifungal agent or salts thereof. The said patent does not disclose or teach cuticle oil composition of Efinaconazole or Tavaborole or Terbinafine or salt thereof.

Currently, the commercially marketed product and product known in the prior art for Efinaconazole or Tavaborole or Terbinafine are available in the form of topical solution, gel, cream, lotion, tincture, ointment or tablet. The commercially marketed product and product known in the prior art suffers from low penetration, difficulty in ease of application at affected site, requires nail lacquer remover, also they may deprive nails from receiving natural light and air necessary for healthy growth of the nails, further gaps may develop between the polished nails and cuticles when left unattended for more than three to four weeks as the nails grow up with passage of time. This results in low efficacy and subsequently poor patient compliance in the treatment of onychomycosis.

Thus, there is an unmet need in the art to provide antifungal cuticle oil composition of Efinaconazole or Tavaborole or Terbinafine or salt thereof which provides better penetration and application at affected site results in better efficacy and patient compliance in the treatment of onychomycosis. The antifungal cuticle oil composition according to present invention also combat fungus of nail, cuticle, nail bed area and skin surrounding nail, protect and nourish the nails which got brittle, inflamed, painful and white to yellow in onychomycosis.

### OBJECTS OF THE INVENTION

It is an object of the present invention is to provide cuticle oil composition of Efinaconazole or Tavaborole or Terbinafine or salt thereof.

It is another object of the present invention is to provide cuticle oil composition of Efinaconazole or Tavaborole or Terbinafine or salt thereof in the treatment of onychomycosis.

It is another object of the present invention is to provide cuticle oil composition of Efinaconazole or Tavaborole or Terbinafine or salt thereof which provides better ease of application at nail region in the treatment of onychomycosis.

It is another object of the present invention is to provide cuticle oil composition of Efinaconazole or Tavaborole or Terbinafine or salt thereof which provides better penetration at affected site results in better efficacy and patient compliance in the treatment of onychomycosis.

It is another object of the present invention is to provide cuticle oil composition of Efinaconazole or Tavaborole or Terbinafine or salt thereof which combat fungus of nail, cuticle, nail bed area and skin surrounding nail, protect and nourish the nails which got brittle, inflamed, painful and white to yellow in onychomycosis.

### SUMMARY OF THE INVENTION

A first aspect of the present invention is to provide pharmaceutical composition of Efinaconazole or Tavaborole or Terbinafine or salt thereof and an oil base; wherein composition is in the form of cuticle oil.

In another aspect of the present invention is to provide oil based composition of Efinaconazole or Tavaborole or Terbinafine or salt thereof for topical administration.

In another aspect of the present invention is to provide oil based composition of Efinaconazole or Tavaborole or Terbinafine or salt thereof for topical administration; preferably in the nail region.

In another aspect of the present invention is to provide process of manufacturing cuticle oil composition comprising Efinaconazole or Tavaborole or Terbinafine or salt thereof along with one or more pharmaceutically acceptable excipient.

In another aspect of the present invention is to provide cuticle oil composition comprising Efinaconazole or Tavaborole or Terbinafine or salt thereof in the treatment of nail disorder.

In another aspect of the present invention is to provide cuticle oil composition comprising Efinaconazole or Tavaborole or Terbinafine or salt thereof in the treatment of nail disorder; preferably in the treatment of onychomycosis.

### DETAIL DESCRIPTION OF THE INVENTION

The present invention relates to pharmaceutical composition of Efinaconazole or Tavaborole or Terbinafine or salt thereof and an oil base; wherein composition is in the form of cuticle oil.

The term cuticle according to present invention is a layer of clear skin located along the bottom edge of finger or toe. This area is known as the nail bed.

The nail bed is the finger tissue or toe tissue that supports the nail. Nails grow continuously throughout a person's life, growing approximately at an average rate of 3 millimeters a month. Fingernails may require 3 to 6 months to re-grow completely, and toenails require approximately 12 to 18 months. Actual growth rate is dependent upon age, gender, season, exercise level, diet, and hereditary factors.

The treatment of fingernail and toe nail problems due to various conditions such as anemia, vitamin or mineral deficiency, onycohrrhexis, onychomycosis, kidney and liver disorders, psoriasis, vascular disease (e.g. Raynaud's disease) and heart disease often result in poor nail growth, vertical trenches, pitting, cracking, horizontal lines, lack of thickness and strength, lack of smoothness and tendency to tear remains a significant problem.

The cuticle oil composition comprising Efinaconazole or Tavaborole or Terbinafine or salt thereof according to present invention is used for topical administration; particularly in the nail region for the treatment of nail disorder.

The nail disorder according to present invention means the any abnormality associated with nail or surrounding region. The cuticle oil composition according to present invention is preferably used in the treatment of onychomycosis.

The onychomycosis is also known as Tinea unguium, is a fungal infection of the nail. The affected region may be the toenails or fingernails. It occurs in about 10 percent of the adult population. The causative organisms in most cases of onychomycosis are dermatophytes. The most common organisms found in onychomycosis are Trichophyton rubrum which is responsible for an estimated 90% of infections and Trichophyton mentagrophytes, which is implicated most commonly in the balance of cases.

The cuticle oil composition comprising Efinaconazole or Tavaborole or Terbinafine or salt thereof and an oil base according to present invention effectively treats fungal infections; wherein Efinaconazole or salt thereof treats onychomycosis by combating Trichophyton rubrum and Trichophyton mentagrophytes through inhibition of fungal lanosterol 14α-demethylase involved in the biosynthesis of ergosterol, a constituent of fungal cell membranes. Tavaborole or salt thereof according to present invention treats onychomycosis by combating Trichophyton rubrum and Trichophyton mentagrophytes through inhibition of fungal protein synthesis. Terbinafine or salt thereof treats onychomycosis by combating Trichophyton rubrum and Trichophyton mentagrophytes through inhibition of biosynthesis of ergosterol, an essential component of fungal cell membrane, via inhibition of squalene epoxidase enzyme.

The cuticle oil composition according to present invention contains any suitable amount of Efinaconazole or Tavaborole or Terbinafine or salt thereof. The concentration of Efinaconazole or salt thereof is at least 1%; preferably ranges from 2 to 20%; more preferably the concentration ranges from 5 to 15% by volume of composition. The concentration of Tavaborole or salt thereof is at least 1%; preferably ranges from 2 to 10%; more preferably the concentration ranges from 3 to 8% by volume of composition. The concentration of Terbinafine or salt thereof is at least 0.2%; preferably ranges from 0.5% to 10%; more preferably the concentration ranges from 0.5% to 5% by volume of composition.

In another aspect of the present invention is to provide cuticle oil composition comprising Efinaconazole or Tavaborole or Terbinafine or salt thereof along with an oil base and one or more pharmaceutically acceptable excipient. The excipients according to present inventions are compatible and acceptable to the nail.

The oil base comprises essential oils and carrier oil or mixtures thereof.

Optionally, the oil base may contain pharmaceutically acceptable excipients selected from the group consisting of, moisturizer, antioxidant, solubilizer, nail bed softener, soothing agent, chelating agent etc.

Optionally the pharmaceutical composition may contain penetration enhancer, fragrance inducer, solvent and preservatives.

The examples of essential oil according to present invention include but not limited to orange oil, rosemary oil, lemon oil, tea tree oil, patchouli oil, lavender oil, eucalyptus oil, peppermint oil, grapefruit oil, carrot seed oil, pumpkin seed oil, papaya seed oil, helichrysum oil, palmarosa oil, lemongrass oil, evening primrose oil, sandalwood oil, myrrh oil, frankincense oil, clove oil, balsam fir oil, wintergreen oil or combinations thereof. The amount of essential oil present in the composition may ranges from about 0.05-10%; preferably about 0.5-6% by volume of the composition.

The examples of carrier oil or base oil according to present invention include but not limited to castor oil, medium-chain triglycerides oil, capmul MCM/glyceryl monocaprylate, almond oil, jojoba oil, coconut oil, grapeseed oil, hemp seed oil, camellia oil, olive oil, wheat germ oil, flax seed oil, argan oil, amla oil, avocado oil, sunflower oil or combinations thereof. The amount of carrier oil present in the composition may ranges from about 15-90%; preferably about 20-85% by volume of composition.

The examples of moisturizer according to present invention include but not limited to medium-chain triglycerides oil, isopropyl myristate, sesame oil, geranium oil, hemp seed oil, chia oil, neem oil, myrrh oil, vitamin E and its oil, aloe-vera, ceramides, glycerin, cocoa butter or shea butter or combinations thereof. The amount of moisturizer present in the composition may ranges from about 10-70%; preferably about 12-60%; more preferably about 15-55% by volume of composition.

The examples of antioxidant according to present invention include but not limited to butylated hydroxytoluene, tocopherols, pomegranate extract, avocado, grapefruit oil, frankincense oil, vitamin E, wheat germ oil, rosemary oil, almond oil, horsetail oil, tea tree oil, sesame oil, ascorbic acid or combinations thereof. The amount of antioxidant present in the composition may ranges from about 0.05-5%; preferably about 0.01-2.5%; more preferably about 0.01-1.0% by volume of composition.

The examples of solubilizer according to present invention include but not limited to ethanol, propylene glycol, propylene glycol monolaurate, PEG-60, isoceteth-20, laureth-23, polyglycerol and its esters, polyethoxylated triglycerides, vitamin E TPGS, water-insoluble lipids (hydrogenated castor oil, corn oil, cottonseed oil, olive oil, peanut oil, peppermint oil, safflower oil, sesame oil, soybean oil, hydrogenated vegetable oils, hydrogenated soybean oil, and medium-chain triglycerides of coconut oil and palm seed oil) or combinations thereof. The amount of solubilizer present in the composition may ranges from about 5-70%; preferably about 7.5-60%; more preferably about 10-50% by volume of composition.

The examples of nail bed softener according to present invention include but not limited to lemon juice, almond oil with ginseng extract, argan oil, rice bran oil, pomegranate seed oil, calendula flowers oil, olive oil, vinegar, vitamin E oil, sunflower oil or combinations thereof.

The examples of soothing agent according to present invention include but not limited to cocoa butter or shea butter, beeswax, aloe-vera gel, sunflower oil, ylang-ylang oil, burdock root extract, sandalwood oil, argan oil, manoi oil, black seed oil, peppermint oil, eucalyptus oil, jojoba oil, almond oil or combinations thereof.

The examples of chelating agent according to present invention include but not limited to calcium disodium, ethylene di amine tetra acetic acid (EDTA), sodium EDTA, calcium, versetamide sodium, calteridol, diethylene triamine penta acetic acid (DTPA) or combinations thereof.

The examples of penetration enhancer according to present invention include but not limited to diethylene glycol monoethyl ether (Transcutol P), turpentine, eucalyptus, peppermint, fennel, almond, sesame, olive, avocado, soybean, raspberry seed, coconut and sea-buckthorn pulp oils, farnesol or combinations thereof. The amount of penetration enhancer present in the composition may ranges from about 0.05-30%; preferably about 0.01-20%; more preferably about 0.5-15% by volume of composition.

The examples of fragrance inducer according to present invention include but not limited to lavender oil, ylang-ylang essential oil, jasmine essential oil, neroli essential oil, sandalwood essential oil, vanilla essential oil, lemongrass essential oil, peppermint essential oil, cedar wood oil, mandarin orange essential oil, rosehip oil, cinnamon oil or combinations thereof. The amount of fragrance inducer present in the composition may ranges from about 0.05-20%; preferably about 0.01-15%; more preferably about 0.5-10% by volume of composition.

The examples of solvent include but not limited to esters, ketones and glycol ethers, aromatic and aliphatic hydrocarbons and alcohols like toluene, benzene, xylene, hexane, heptanes, naphthas, light petroleum ether, n-butyl acetate, ethyl acetate, alcohol, acetone, methyl glycol acetate, methyl ethyl ketone, methyl isobutyl ketone, methyl acetate, toluene, isopropanol or combination thereof. The amount of solvent present in the composition may ranges from about 5-90%; preferably about 10-70%; more preferably about 15-50% by volume of composition.

The examples of preservatives include but not limited to chorocresol, phenyl mercuric nitrate, benzyl alcohol, benzoic acid and its salts, boric acid, methyl paraben, propyl paraben, trihydrate and anhydrous sodium acetate, chlorhexidine, formaldehyde, glutaraldehyde, imidazolidinyl urea, triclosan, benzalkonium chloride and chloroxylenol.

In another aspect of the present invention is to provide process of manufacturing cuticle oil composition comprising Efinaconazole or Tavaborole or Terbinafine or salt thereof along with one or more pharmaceutically acceptable excipient.

The process of manufacturing cuticle oil composition according to the present invention involves step of mixing Efinaconazole or Tavaborole or Terbinafine or salt thereof, essential oil, carrier oil along with one or more pharmaceutically acceptable excipient selected from the group consisting of moisturizer, antioxidants, solubilizer, nail bed softener, soothing agent or chelating agent.

The process of manufacturing cuticle oil composition as well as the concentration or amount of Efinaconazole or Tavaborole or Terbinafine or salt thereof, oil and one or more pharmaceutically acceptable excipient has been optimized in such way that formed cuticle oil provides better ease of application, better penetration, soothing effect, moisturizing effect, strengthening effect, at affected site; therefore results in the maximum therapeutic efficacy with better patient compliance in the treatment of Onychomycosis.

The antifungal cuticle oil composition according to present invention combat fungus of nail, cuticle, nail bed area and skin surrounding nail, protect and nourish the nails which got brittle, inflamed, painful and white to yellow in onychomycosis. The cuticle oil composition comprising Efinaconazole or Tavaborole or Terbinafine or salt thereof according to present invention effectively combats Trichophyton rubrum, Trichophyton mentagrophytes which are main causative organisms in the onychomycosis.

The cuticle oil composition comprising Efinaconazole or Tavaborole or Terbinafine or salt thereof according to present invention were evaluated for parameters like color, odour, taste, rancidity, assay and found to be satisfactory.

The cuticle oil composition comprising Efinaconazole or Tavaborole or Terbinafine or salt thereof according to present invention packaged into the suitable container like glass bottles with a dropper, glass bottles with a brush applicator, cuticle oil dispensing pen, cotton ball, plastic squeeze bottle, bottles or any suitable packaging material.

### EXAMPLES

The following Examples are provided solely for illustrative purposes and are not meant to limit the invention in any way.

### Example 1

| Sr. No. | Ingredients | (mg/ml) | | % | |
|---|---|---|---|---|---|
| 1. | Tavaborole | 50.0 | | 5.0 | w/v |
| 2. | Butylated hydroxytoluene | 1.00 | | 0.1 | w/v |
| 3. | Capmul MCM/Glyceryl | 0.4 | ml | 40 | v/v |
| | monocaprylate | | | | |

| | | | |
|---|---|---|---|
| 4. | Propylene glycol | q.s. | q.s. |

| | | | |
|---|---|---|---|
| Total weight | 1 | ml | 100.00 |

### Manufacturing Process:

1. Tavaborole was dissolved in part quantity of propylene glycol using stirrer at room temperature.
2. Capmul MCM/Glyceryl monocaprylate was added to step 1, mixed using stirrer.
3. Butylated hydroxytoluene was dissolved in step 2 to form clear oil.
4. Volume was made up to required quantity using propylene glycol.

### Example 2

| Sr. No. | Ingredients | (mg/ml) | | % | |
|---|---|---|---|---|---|
| 1. | Tavaborole | 50.0 | | 5.0 | w/v |
| 2. | Butylated hydroxytoluene | 1.00 | | 0.1 | w/v |
| 3. | Ethanol | 0.2 | ml | 20.0 | v/v |

| | | | |
|---|---|---|---|
| 4. | Castor oil | q.s. | q.s. |

| | | | |
|---|---|---|---|
| Total weight | 1 | ml | 100.00 |

### Manufacturing Process:

1. Tavaborole was dissolved in ethanol using stirrer at room temperature.
2. Butylated hydroxytoluene was dissolved in step 1 to form clear oil.
3. Castor oil was mixed with step 2, and volume was made up to required quantity using castor oil.

### Example 3

| Sr. No. | Ingredients | (mg/ml) | | % | |
|---|---|---|---|---|---|
| 1. | Tavaborole | 50.0 | | 5.0 | w/v |
| 2. | Butylated hydroxytoluene | 1.00 | | 0.1 | w/v |
| 3. | Ethanol | 0.12 | ml | 12.0 | v/v |
| 4. | Medium-chain triglycerides oil | 0.20 | ml | 20.0 | v/v |
| 5. | Diethylene glycol monoethyl | 0.05 | ml | 5.0 | v/v |
| | ether (Transcutol P) | | | | |

| | | | |
|---|---|---|---|
| 6. | Castor oil | q.s. | q.s. |

| | | | |
|---|---|---|---|
| Total weight | 1 | ml | 100.00 |

### Manufacturing Process:

1. Tavaborole was dissolved in ethanol using stirrer at room temperature.
2. Butylated hydroxytoluene, medium-chain triglycerides oil, diethylene glycol monoethyl ether and castor oil were mixed with step 1 and volume was made up to required quantity using castor oil.

### Example 4

| Sr. No. | Ingredients | (mg/ml) | | % | |
|---|---|---|---|---|---|
| 1. | Tavaborole | 50.0 | | 5.0 | w/v |
| 2. | Butylated hydroxytoluene | 1.0 | | 0.1 | w/v |
| 3. | Ethanol | 0.12 | ml | 12.0 | v/v |
| 4. | Isopropyl myristate | 0.5 | ml | 50.0 | v/v |
| 5. | Diethylene glycol monoethyl | 0.05 | ml | 5.0 | v/v |
| | ether (Transcutol P) | | | | |

| | | | |
|---|---|---|---|
| 6. | Castor oil | q.s. | q.s. |

| | | | |
|---|---|---|---|
| Total weight | 1 | ml | 100.00 |

### Manufacturing Process:

1. Tavaborole was dissolved in ethanol using stirrer at room temperature.
2. Butylated hydroxytoluene, isopropyl myristate, diethylene glycol monoethyl ether and castor oil were mixed with step 1 and volume was made up to required quantity using castor oil.

### Example 5

| Sr. No. | Ingredients | (mg/ml) | | % | |
|---|---|---|---|---|---|
| 1. | Tavaborole | 50.0 | | 5.0 | w/v |
| 2. | Butylated hydroxytoluene | 1.00 | | 0.1 | w/v |
| 3. | Ethanol | 0.2 | ml | 20.0 | v/v |

| | | | | |
|---|---|---|---|---|
| 4. | Lavender oil | 1 | 0.1 | w/v |

| | | | |
|---|---|---|---|
| 5. | Castor oil | q.s. | q.s. |

| | | | |
|---|---|---|---|
| Total weight | 1 | ml | 100.00 |

### Manufacturing Process:

1. Tavaborole was dissolved in ethanol using stirrer at room temperature.
2. Butylated hydroxytoluene was dissolved in step 1 to form clear oil.
3. Lavender oil was mixed with step 2.
4. Castor oil was mixed with step 3, and volume was made up to required quantity using castor oil.

### Example 6

| Sr. No. | Ingredients | (mg/ml) | % | |
|---|---|---|---|---|
| 1. | Efinaconazole | 100.00 | 10.0 | w/v |
| 2. | Propylene glycol | 300.00 | 30.00 | v/v |
| | monolaurate (type II) | | | |
| 3. | Diethylene glycol monoethyl | 50.00 | 5.0 | v/v |
| | ether | | | |
| 4. | Butylated hydroxytoluene | 1.00 | 0.1 | w/v |

| | | | |
|---|---|---|---|
| 5. | Olive Oil | q.s | q.s |

| | | |
|---|---|---|
| Total weight | 1 ml | 100.00 |

### Manufacturing Process:

1. Efinaconazole was dissolved in Propylene glycol monolaurate (type II) using stirrer at room temperature.
2. Diethylene glycol monoethyl ether was added to step 1, mixed using stirrer.
3. Butylated hydroxytoluene was dissolved in step 2 to form clear solution.
4. Volume was made up to required quantity using Olive oil.

### Example 7

| | Ingredients | (mg/ml) | % | |
|---|---|---|---|---|
| 1. | Efinaconazole | 100.00 | 10.00 | w/v |
| 2. | Propylene glycol | 300.00 | 30.00 | v/v |
| | monolaurate (type II) | | | |
| 3. | Diethylene glycol monoethyl | 50.00 | 5.00 | v/v |
| | ether | | | |
| 4. | Butylated hydroxytoluene | 1.00 | 0.10 | w/v |
| 5. | Nitrocellulose | 100.00 | 10.00 | w/v |
| 6. | Ethyl acetate | 200.00 | 20.00 | v/v |
| 7. | Lavender Oil | 50.00 | 5.00 | v/v |

| | | | |
|---|---|---|---|
| 8. | Sweet almond Oil | q.s | q.s |

| | | |
|---|---|---|
| Total weight | 1 ml | 100.00 |

### Manufacturing Process:

1. Efinaconazole was dissolved in Propylene glycol monolaurate (type II) using stirrer at room temperature.
2. Diethylene glycol monoethyl ether was added to step 1, mixed using stirrer.
3. Butylated hydroxytoluene was dissolved in step 2 to form clear solution.
4. In a separate container, nitrocellulose was dissolved in ethyl acetate and the resultant viscous solution added to solution of step 2 to form clear solution.
5. Lavender oil added to solution of step 4.
6. Volume was made up to required quantity using Sweet almond oil.

### Example 8

| Sr. No. | Ingredients | (mg/ml) | % | |
|---|---|---|---|---|
| 1. | Efinaconazole | 100.00 | 10.00 | w/v |
| 2. | Propylene glycol | 200.00 | 20.00 | v/v |
| | monolaurate (type II) | | | |
| 3. | Diethylene glycol monoethyl | 50.00 | 5.00 | v/v |
| | ether | | | |
| 4. | Butylated hydroxytoluene | 1.00 | 0.10 | w/v |
| 5. | Nitrocellulose | 100.00 | 10.00 | w/v |
| 6. | Ethyl acetate | 200.00 | 20.00 | v/v |
| 7. | Lavender Oil | 50.00 | 5.00 | v/v |

| | | | |
|---|---|---|---|
| 8. | Sweet almond Oil | q.s | q.s |

| | | |
|---|---|---|
| Total weight | 1 ml | 100.00 |

### Manufacturing Process:

1. Efinaconazole was dissolved in Propylene glycol monolaurate (type II) using stirrer at room temperature.
2. Diethylene glycol monoethyl ether was added to step 1, mixed using stirrer.
3. Butylated hydroxytoluene was dissolved in step 2 to form clear solution.
4. In a separate container, nitrocellulose was dissolved in ethyl acetate and the resultant viscous solution added to solution of step 2 to form clear solution.
5. Lavender oil added to solution of step 4.
6. Volume was made up to required quantity using Sweet almond oil.

### Example 9

| Sr. No. | Ingredients | (mg/ml) | % | |
|---|---|---|---|---|
| 1. | Efinaconazole | 100.00 | 10.00 | w/v |
| 2. | Propylene glycol | 300.00 | 30.00 | v/v |
| | monolaurate (type II) | | | |
| 3. | Diethylene glycol monoethyl | 100.00 | 10.00 | v/v |
| | ether | | | |
| 4. | Butylated hydroxytoluene | 1.00 | 0.10 | w/v |
| 5. | Nitrocellulose | 100.00 | 10.00 | w/v |
| 6. | Ethyl acetate | 200.00 | 20.00 | v/v |
| 7. | Lavender Oil | 50.00 | 5.00 | v/v |

| | | | |
|---|---|---|---|
| 8. | Sweet almond Oil | q.s | q.s |

| | | |
|---|---|---|
| Total weight | 1 ml | 100.00 |

Manufacturing Process:

1. Efinaconazole was dissolved in Propylene glycol monolaurate (type II) using stirrer at room temperature.

2. Diethylene glycol monoethyl ether was added to step 1, mixed using stirrer.

3. Butylated hydroxytoluene was dissolved in step 2 to form clear solution.

4. In a separate container, nitrocellulose was dissolved in ethyl acetate and the resultant viscous solution added to solution of step 2 to form clear solution.

5. Lavender oil added to solution of step 4.

6. Volume was made up to required quantity using Sweet almond oil.

### Example 10

| Sr. No. | Ingredients | (mg/ml) | % | |
|---|---|---|---|---|
| 1. | Efinaconazole | 100.00 | 10.00 | w/v |
| 2. | Propylene glycol | 300.00 | 30.00 | v/v |
| | monolaurate (type II) | | | |
| 3. | Diethylene glycol monoethyl | 50.00 | 5.00 | v/v |
| | ether | | | |
| 4. | Butylated hydroxytoluene | 1.00 | 0.10 | w/v |
| 5. | Nitrocellulose | 100.00 | 10.00 | w/v |
| 6. | Ethyl acetate | 200.00 | 20.00 | v/v |
| 7. | Lavender Oil | 50.00 | 5.00 | v/v |

| | | | |
|---|---|---|---|
| 8. | Sesame Oil | q.s | q.s |

| | | |
|---|---|---|
| Total weight | 1 ml | 100.00 |

### Manufacturing Process:

1. Efinaconazole was dissolved in Propylene glycol monolaurate (type II) using stirrer at room temperature.
2. Diethylene glycol monoethyl ether was added to step 1, mixed using stirrer.
3. Butylated hydroxytoluene was dissolved in step 2 to form clear solution.
4. In a separate container, nitrocellulose was dissolved in ethyl acetate and the resultant viscous solution added to solution of step 2 to form clear solution.
5. Lavender oil added to solution of step 4.
6. Volume was made up to required quantity using Sesame oil.

### Example 11

| Sr. No. | Ingredients | (mg/ml) | % | |
|---|---|---|---|---|
| 1. | Efinaconazole | 100.00 | 10.00 | w/v |
| 2. | Propylene glycol | 300.00 | 30.00 | v/v |
| | monolaurate (type II) | | | |
| 3. | Diethylene glycol monoethyl | 50.00 | 5.00 | v/v |
| | ether | | | |
| 4. | Butylated hydroxytoluene | 1.00 | 0.10 | w/v |
| 5. | Nitrocellulose | 100.00 | 10.00 | w/v |
| 6. | Ethyl acetate | 200.00 | 20.00 | v/v |
| 7. | Lavender Oil | 50.00 | 5.00 | v/v |

| | | | |
|---|---|---|---|
| 8. | Olive Oil | q.s | q.s |

| | | |
|---|---|---|
| Total weight | 1 ml | 100.00 |

### Manufacturing Process:

1. Efinaconazole was dissolved in Propylene glycol monolaurate (type II) using stirrer at room temperature.
2. Diethylene glycol monoethyl ether was added to step 1, mixed using stirrer.
3. Butylated hydroxytoluene was dissolved in step 2 to form clear solution.
4. In a separate container, nitrocellulose was dissolved in ethyl acetate and the resultant viscous solution added to solution of step 2 to form clear solution.
5. Lavender oil added to solution of step 4.
6. Volume was made up to required quantity using Olive oil.

### Example 12

| Sr. No. | Ingredients | (mg/ml) | | % | |
|---|---|---|---|---|---|
| 1. | Terbinafine | 10.0 | | 1.0 | w/v |
| 2. | Butylated hydroxytoluene | 1.00 | | 0.1 | w/v |
| 3. | Capmul MCM/Glyceryl | 0.4 | ml | 40 | v/v |
| | monocaprylate | | | | |

| | | | |
|---|---|---|---|
| 4. | Propylene glycol | q.s. | q.s. |

| | | | |
|---|---|---|---|
| Total weight | 1 | ml | 100.00 |

### Manufacturing Process:

1. Terbinafine was dissolved in part quantity of propylene glycol using stirrer at room temperature.
2. Capmul MCM/Glyceryl monocaprylate was added to step 1, mixed using stirrer.
3. Butylated hydroxytoluene was dissolved in step 2 to form clear oil.
4. Volume was made up to required quantity using propylene glycol.

### Example 13

| Sr. No. | Ingredients | (mg/ml) | | % | |
|---|---|---|---|---|---|
| 1. | Terbinafine | 10.0 | | 1.0 | w/v |
| 2. | Butylated hydroxytoluene | 1.00 | | 0.1 | w/v |
| 3. | Ethanol | 0.2 | ml | 20.0 | v/v |

| | | | |
|---|---|---|---|
| 4. | Castor oil | q.s. | q.s. |

| | | | |
|---|---|---|---|
| Total weight | 1 | ml | 100.00 |

### Manufacturing Process:

1. Terbinafine was dissolved in ethanol using stirrer at room temperature.
2. Butylated hydroxytoluene was dissolved in step 1 to form clear oil.
3. Castor oil was mixed with step 2, and volume was made up to required quantity using castor oil.

### Example 14

| Sr. No. | Ingredients | (mg/ml) | | % | |
|---|---|---|---|---|---|
| 1. | Terbinafine | 10.0 | | 1.0 | w/v |
| 2. | Butylated hydroxytoluene | 1.00 | | 0.1 | w/v |
| 3. | Ethanol | 0.12 | ml | 12.0 | v/v |
| 4. | Medium-chain triglycerides | 0.20 | ml | 20.0 | v/v |
| | oil | | | | |
| 5. | Diethylene glycol monoethyl | 0.05 | ml | 5.0 | v/v |
| | ether (Transcutol P) | | | | |

| | | | |
|---|---|---|---|
| 6. | Castor oil | q.s. | q.s. |

| | | | |
|---|---|---|---|
| Total weight | 1 | ml | 100.00 |

### Manufacturing Process:

1. Terbinafine was dissolved in ethanol using stirrer at room temperature.
2. Butylated hydroxytoluene, medium-chain triglycerides oil, diethylene glycol monoethyl ether and castor oil were mixed with step 1 and volume was made up to required quantity using castor oil.

### Example 15

| Sr. No. | Ingredients | (mg/ml) | | % | |
|---|---|---|---|---|---|
| 1. | Terbinafine | 10.0 | | 1.0 | w/v |
| 2. | Butylated hydroxytoluene | 1.0 | | 0.1 | w/v |
| 3. | Ethanol | 0.12 | ml | 12.0 | v/v |
| 4. | Isopropyl myristate | 0.5 | ml | 50.0 | v/v |
| 5. | Diethylene glycol monoethyl | 0.05 | ml | 5.0 | v/v |
| | ether (Transcutol P) | | | | |

| | | | |
|---|---|---|---|
| 6. | Castor oil | q.s. | q.s. |

| | | | |
|---|---|---|---|
| Total weight | 1 | ml | 100.00 |

### Manufacturing Process:

1. Terbinafine was dissolved in ethanol using stirrer at room temperature.
2. Butylated hydroxytoluene, isopropyl myristate, diethylene glycol monoethyl ether and castor oil were mixed with step 1 and volume was made up to required quantity using castor oil.

### Example 16

| Sr. No. | Ingredients | (mg/ml) | | % | |
|---|---|---|---|---|---|
| 1. | Terbinafine | 10.0 | | 1.0 | w/v |
| 2. | Butylated hydroxytoluene | 1.00 | | 0.1 | w/v |
| 3. | Ethanol | 0.2 | ml | 20.0 | v/v |

| | | | | |
|---|---|---|---|---|
| 4. | Lavender oil | 1 | 0.1 | w/v |

| | | | |
|---|---|---|---|
| 5. | Castor oil | q.s. | q.s. |

| | | | |
|---|---|---|---|
| Total weight | 1 | ml | 100.00 |

### Manufacturing Process:

1. Terbinafine was dissolved in ethanol using stirrer at room temperature.
2. Butylated hydroxytoluene was dissolved in step 1 to form clear oil.
3. Lavender oil was mixed with step 2.
4. Castor oil was mixed with step 3, and volume was made up to required quantity using castor oil.

1. A cuticle oil pharmaceutical composition comprising i) antifungal agent selected from Efinaconazole or Tavaborole or Terbinafine or salt thereof; and ii) an oil base.

2. The pharmaceutical composition according to claim 1, wherein the oil base comprises carrier oil or essential oil or mixtures thereof.

3. The pharmaceutical composition according to claim 1, wherein the oil base further comprises one or more pharmaceutically acceptable excipients.

4. The pharmaceutical composition according to claim 1, is in the form of topical composition.

5. The pharmaceutical composition according to claim 1, is in the form of topical nail composition.

6. The pharmaceutical composition according to claim 1, wherein concentration of Efinaconazole or salt thereof ranges from 5% to 15% by volume of the composition.

7. The pharmaceutical composition according to claim 1, wherein concentration of Tavaborole or salt thereof ranges from 3% to 8% by volume of the composition.

8. The pharmaceutical composition according to claim 1, wherein concentration of Terbinafine or salt thereof ranges from 0.5% to 5% by volume of the composition.

9. The pharmaceutical composition according to claim 1, wherein concentration of carrier oil ranges from 20-85% by volume of the composition and concentration of essential oil ranges from 0.5-6% by volume of the composition.

10. The pharmaceutical composition according to claim 1, wherein carrier oil is selected from the group consisting of castor oil, almond oil, olive oil, medium-chain triglycerides oil, capmul MCM/glyceryl monocaprylate, jojoba oil, coconut oil, grapeseed oil, hemp seed oil, camellia oil, wheat germ oil, flax seed oil, argan oil, amla oil, avocado oil, sunflower oil or mixture thereof.

11. The pharmaceutical composition according to claim 1, wherein essential oil is selected from the group consisting of lavender oil, orange oil, rosemary oil, lemon oil, tea tree oil, patchouli oil, eucalyptus oil, peppermint oil, grapefruit oil, carrot seed oil, pumpkin seed oil, papaya seed oil, helichrysum oil, palmarosa oil, lemongrass oil, evening primrose oil, sandalwood oil, myrrh oil, frankincense oil, clove oil, balsam fir oil, wintergreen oil or mixture thereof.

12. The pharmaceutical composition according to claim 3, wherein the one or more pharmaceutically acceptable excipients is selected from the group consisting of moisturizer, antioxidants, penetration enhancer, fragrance inducer, solubilizer, solvent, nail bed softener, soothing agent, chelating agent and preservatives.

13. The cuticle oil pharmaceutical composition of antifungal agent according to claim 1 for the treatment of onychomycosis.
